(19) United States
(12) Patent Application Publication
(10) Pub. No.: US 2021/0052788 A1
(43) Pub. Date: Feb. 25, 2021

(54) A SYSTEM FOR MANAGING INSTILLATION THERAPY ON MULTIPLE WOUNDS WITH A SINGLE FLUID SOURCE

(71) Applicant: KCI LICENSING, INC., San Antonio, TX (US)

(72) Inventors: Benjamin A. PRATT, Poole (GB); Justin A. LONG, Bournemouth (GB)

(21) Appl. No.: 17/040,377

(22) PCT Filed: Mar. 13, 2019
(86) PCT No.: PCT/US2019/022105

§ 371 Date: Sep. 22, 2020

Related U.S. Application Data
(60) Provisional application No. 62/649,708, filed on Mar. 29, 2018.

(51) Int. Cl. A61M 1/00; A61F 13/02

(52) U.S. Cl. CPC A61M 1/0035; A61F 2013/00114; A61M 1/0088; A61F 13/0216

(57) ABSTRACT

A valve arrangement for administering an instillation and/or negative pressure therapy through at least a first wound dressing and a second wound dressing includes a valve arrangement including a first flow path, a first valve, a second flow path, and a second valve. The first flow path extends between a source port and a first wound dressing port structured for fluid communication with the first wound dressing. The first valve is positioned in the first flow path. The second flow path extends between the source port and a second wound dressing port structured for fluid communication with the second wound dressing. The second valve is positioned in the second flow path.

### CROSS-REFERENCE TO RELATED APPLICATIONS

The present application claims the benefit, under 35 USC § 119(e), of the filing of U.S. Provisional Patent Application 62/649,708, titled “A SYSTEM FOR MANAGING INSTILLATION THERAPY ON MULTIPLE WOUNDS WITH A SINGLE FLUID SOURCE,” filed Mar. 29, 2018. This provisional application is incorporated herein by reference in its entirety for all purposes.

### BACKGROUND

The present disclosure relates generally to a wound therapy system, and more particularly to a wound therapy system structured to administer negative pressure wound therapy and/or instillation therapy to more than one wound site on a patient.

Negative pressure wound therapy (NPWT) is a type of wound therapy that involves applying negative pressure (pressure lower than atmospheric pressure) to a wound site to promote wound healing. Some NPWT systems include a pump which operates to maintain the wound site at negative pressure by removing wound exudate from the wound site. The wound exudate is typically routed to a canister or other container fluidly connected to the pump where the wound exudate is stored until emptied by a user.

Instillation therapy is a type of wound therapy that involves applying a therapeutic fluid (e.g. a saline solution, a prescribed solution, an antibiotic, a cleaning fluid etc.) to a wound site to promote wound healing and granulation, prevent the wound from drying out, prevent the wound from becoming infected by bacteria and/or treat an infected wound site. Some instillation systems include an instillation fluid container and an instillation pump for providing instillation fluid to the wound site. Instillation therapy can be used in conjunction with NPWT or can be used separately.

A patient may have multiple wounds that require different amounts of NPWT and/or instillation therapy (e.g., due to differences in wound size and/or wound conditions, etc.). It is generally difficult to administer differing amounts of NPWT and/or instillation therapy to multiple wound sites on the patient using a single negative pressure source and/or a single instillation source. Accordingly, for patients having multiple wounds that require NPWT and/or instillation therapy, a different NPWT and/or instillation system is generally used for each of the wounds so that the NPWT and/or instillation therapy administered to each of the wound sites can be customized based on the conditions of the wound site (e.g. wound site size, wound dressing volume, wound site dryness, etc.). Accordingly, providing NPWT and/or instillation therapy to a patient with multiple wounds can be complex to setup and to manage. It would be desirable to administer a wound therapy system that overcomes these and other disadvantages of conventional fluid delivery and removal systems.

### SUMMARY

One implementation of the present disclosure is a valve arrangement for administering an instillation and/or negative pressure therapy through at least a first wound dressing and a second wound dressing. The valve arrangement includes an instillation source port, a negative pressure source port, a first instillation flow path, a second instillation flow path, a first negative pressure flow path, and a second negative pressure flow path. The instillation source port is structured to engage a instillation fluid source. The negative pressure source port is structured to engage a negative pressure source. The first instillation flow path includes a first instillation valve. The first instillation flow path extends between the instillation source port and a first instillation wound dressing port structured for fluid communication with the first wound dressing. The second instillation flow path includes a second instillation valve. The second instillation flow path extends between the instillation source port and a second instillation wound dressing port structured for fluid communication with the second wound dressing. The first negative pressure flow path includes a first negative pressure valve. The first negative pressure flow path extends between the negative pressure source port and a first negative pressure wound dressing port structured for fluid communication with the first wound dressing. The second negative pressure flow path includes a second negative pressure valve. The second negative pressure flow path extends between the negative pressure source port and a second negative pressure wound dressing port structured for fluid communication with the second wound dressing.

Another implementation of the present disclosure is a valve arrangement for administering an instillation and/or negative pressure therapy through at least a first wound dressing and a second wound dressing. The valve arrangement includes a first flow path, a first valve, a second flow path, and a second valve. The first flow path extends between a source port and a first wound dressing port structured for fluid communication with the first wound dressing. The first valve is positioned in the first flow path. The second flow path extends between the source port and a second wound dressing port structured for fluid communication with the second wound dressing. The second valve is positioned in the second flow path.

Another implementation of the present disclosure is a system including a fluid source, a negative pressure source, an instillation valve arrangement, and a negative pressure valve arrangement. The fluid source is structured to dispense an instillation fluid. The negative pressure source is structured to generate a negative pressure. The instillation valve arrangement includes a first instillation flow path, a first installation valve, a second installation flow path, and a second instillation valve. The first instillation flow path extends between an instillation source port structured to engage the fluid source and a first wound dressing port structured to engage a first wound dressing. The first instillation valve is positioned in the first instillation flow path. The second instillation flow path extends between the instillation source port and a second wound dressing port structured to engage a second wound dressing. The second instillation valve is positioned in the second instillation flow path. The negative pressure valve arrangement includes a first negative pressure flow path, a first negative pressure valve, a second negative pressure flow path, and a second negative pressure valve. The first negative pressure flow path extends between a negative pressure source port structured to engage the negative pressure source and a first wound dressing port structured to engage the first wound dressing. The first negative pressure valve is positioned in the first negative pressure flow path. The second negative pressure flow path extends between the negative pressure source port and a second wound dressing port structured to engage the second wound dressing. The second negative pressure valve is positioned in the second negative pressure flow path.

Those skilled in the art will appreciate that the summary is illustrative only and is not intended to be in any way limiting. Other aspects, inventive features, and advantages of the devices and/or processes described herein, as defined solely by the claims, will become apparent in the detailed description set forth herein and taken in conjunction with the accompanying drawings.

### BRIEF DESCRIPTION OF THE DRAWINGS

FIG. 1 is a schematic representation of a wound treatment system according to an exemplary embodiment.

FIG. 2 is a schematic representation of a wound treatment system according to another exemplary embodiment.

FIG. 3 is a schematic representation of an instillation device in a priming configuration according to another exemplary embodiment.

FIG. 4 is a schematic representation of the instillation device of FIG. 3 in an instillation configuration.

FIG. 5 is a schematic representation of a valve arrangement for use with the wound treatment system of FIGS. 1 and/or 2 according to an exemplary embodiment.

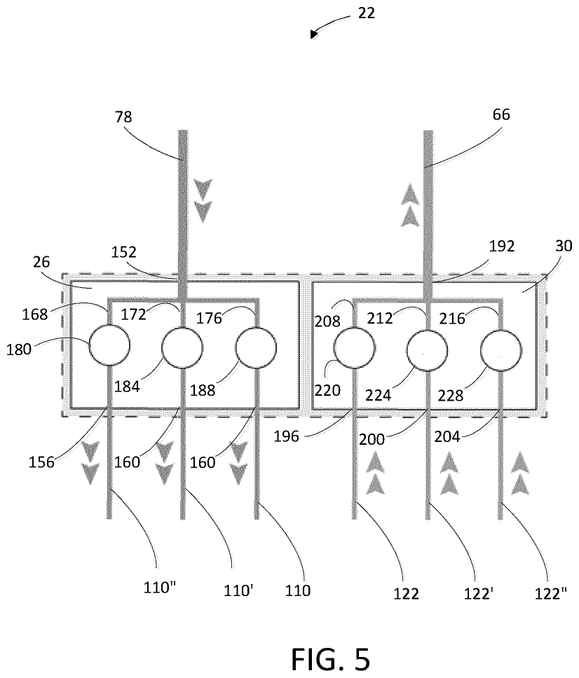

FIG. 6 is a schematic representation of a valve arrangement for use with the wound treatment system of FIGS. 1 and/or 2 according to another exemplary embodiment.

FIG. 7 is a schematic representation of a controller of the wound treatment system of FIGS. 1 and/or 2 according to an exemplary embodiment.

FIG. 8 is a schematic representation of a method for using the wound treatment system of FIGS. 1 and/or 2.

### DETAILED DESCRIPTION

### Overview

Referring generally to the FIGURES, a wound therapy system for administering instillation therapy and/or negative pressure wound therapy (NPWT) to more than one wound on a patient is shown, according to various embodiments. More specifically, the wound therapy system may include an instillation system, a NPWT system, a valve arrangement (e.g., fluid distribution device, valve block or valve blocks), and a plurality of wound dressings. As depicted in the figures, the plurality of wound dressings includes three wound dressings. However, other embodiments of the wound therapy system may include more or fewer wound dressings.

The instillation system can be structured to store and dispense an instillation fluid (e.g., a cleansing fluid, a prescribed fluid, etc.) for delivery to at least one of the wound sites. The NPWT system can be structured to administer negative pressure generated by a negative pressure source to at least one of the wound sites. In some embodiments, a removed fluid container can be positioned upstream of the negative pressure source for receiving and storing a fluid removed from the wound site (e.g., wound exudate, previously-delivered instillation fluid, etc). The valve arrangement is structured to administer selective fluid communication between the instillation system, the NPWT system, and the plurality of wound dressings to administer instillation therapy to the wound site, administer NPWT to the wound site, and/or administer a combination of instillation therapy and NPWT to the wound site.

As illustrated in the figures, the valve arrangement includes an instillation wound dressing portion for providing instillation therapy to the plurality of wound sites and a NPWT portion for providing NPWT to the plurality of wound sites. The instillation portion includes an instillation source port structured to engage the instillation system and a plurality of instillation wound dressing ports for providing instillation fluid to the plurality of wound dressings. A plurality of instillation flow paths extends between the instillation source port and each of the plurality of instillation wound dressing ports. A valve is positioned in each of the plurality of instillation flow paths. The valves are independently positionable to control an amount of instillation fluid dispensed each wound dressing of the plurality of wound dressings. In some embodiments, the valves can be manually positioned. In other embodiments, the valves can be positioned by a controller. In such embodiments, the controller can position the valves based on information indicative of a status each wound dressing of the plurality of wound dressings sensed by a sensor positioned in each wound dressing of the plurality of wound dressings.

The NPWT portion is structured similarly to the instillation wound dressing portion. The NPWT portion includes an NPWT source port structured to engage the NPWT system, and a plurality of NPWT ports for providing negative pressure to the plurality of wound dressings. A plurality of NPWT flow paths extends between the NPWT source port and each of the plurality of NPWT ports. A valve is positioned in each of the plurality of NPWT flow paths. The valves are independently positionable to control an amount of negative pressure administered to each wound dressing of the plurality of wound dressings. In some embodiments, the valves can be manually positioned. In other embodiments, the valves can be positioned by a controller. In such embodiments, the controller can position the valves based on information indicative of a status of each wound dressing of the plurality of wound dressings sensed by the sensor positioned in each wound dressing of the plurality of wound dressings.

Accordingly, the valve arrangement is structured to independently administer instillation therapy and/or NPWT for each of the plurality of wound dressings using a single instillation system and a single NPWT system by providing (1) separate flow paths with independently controllable valves between the instillation system and each of the plurality of wound dressings, and (2) separate flow paths with independently controllable valves between the plurality of wound dressings and the negative pressure system. For example, a first wound dressing of the plurality of wound dressings can receive instillation fluid by opening a first instillation valve of the plurality of instillation valves and closing a first negative pressure valve of the plurality of negative pressure valves. A second wound dressing of the plurality of wound dressings can receive negative pressure by closing a second instillation valve of the plurality of instillation valves and opening a first negative pressure valve of the plurality of negative pressure valves. A third wound dressing of the plurality of wound dressings can receive both instillation fluid and negative pressure by opening a third instillation valve of the plurality of instillation valves and opening a first negative pressure valve of the plurality of negative pressure valves.

Additional features and advantages of the wound therapy system are described in detail below.

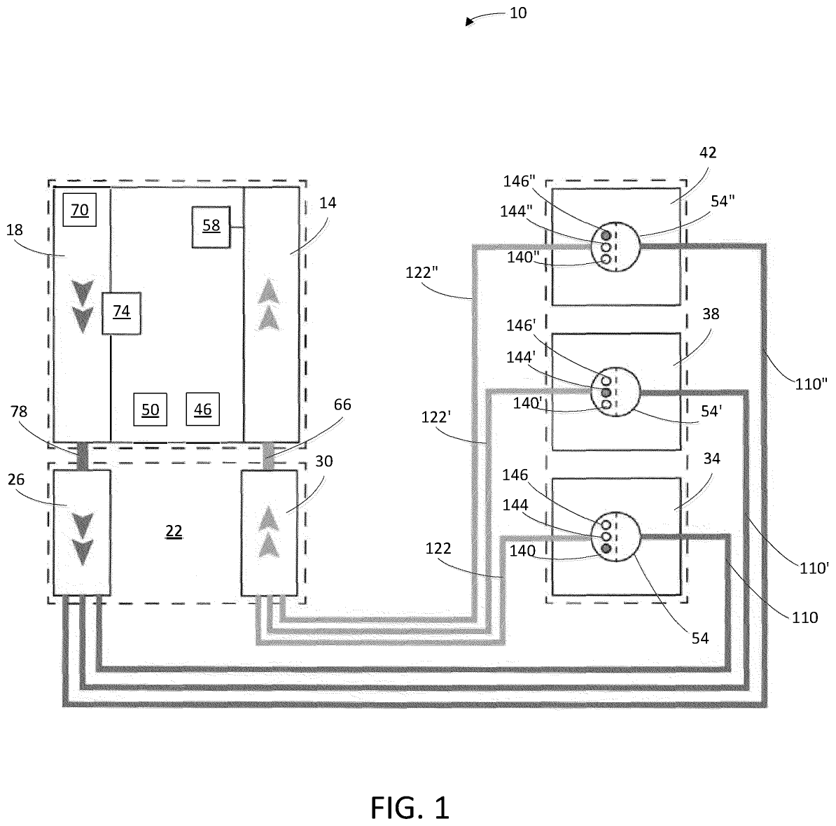

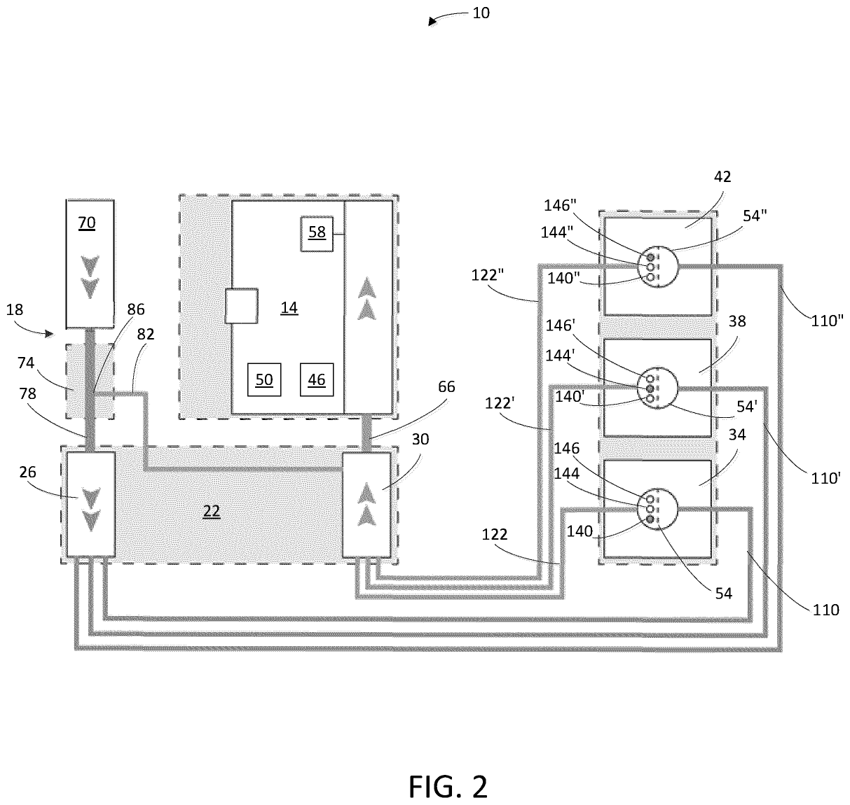

Referring now to FIGS. 1-2, a wound therapy system 10 is shown, according to an exemplary embodiment. The wound therapy system 10 includes a negative pressure wound therapy (NPWT) system 14, an instillation system 18, a valve arrangement (e.g., fluid distribution device, valve block or valve blocks) 22, and a plurality of wound dressings. In the illustrated embodiments, the valve arrangement 22 includes an instillation valve arrangement 26 and a NPWT valve arrangement. In the illustrated embodiment, the plurality of wound dressings includes a first wound dressing 34, a second wound dressing 38, and a third wound dressing 42. In other embodiments, the plurality of wound dressings could include more or fewer wound dressings.

### Negative Pressure Wound Treatment System

The NPWT system 14 includes a suction source or a negative pressure source 58, a collection chamber (not shown), a control system 46, and a power source 50. The negative pressure source 58 is structured to generate negative pressure that can be administered to the wound sites through the wound dressings 34, 38, 42. The negative pressure source 58 is in fluid communication with the valve arrangement 22 via a negative pressure conduit 66. In some embodiments, the collection chamber can be positioned along the negative pressure conduit 66 between the wound dressings 34, 38, 42 and the negative pressure source 58 for collecting fluid (e.g. exudate and/or instillation fluids) from the wound dressings 34, 38, 42. In such an embodiment, the negative pressure conduit 66 includes a structure (e.g., a one-direction valve) for preventing the fluid from entering the negative pressure source 58. In some embodiments, the negative pressure source 58 may be a pump.

### Instillation System

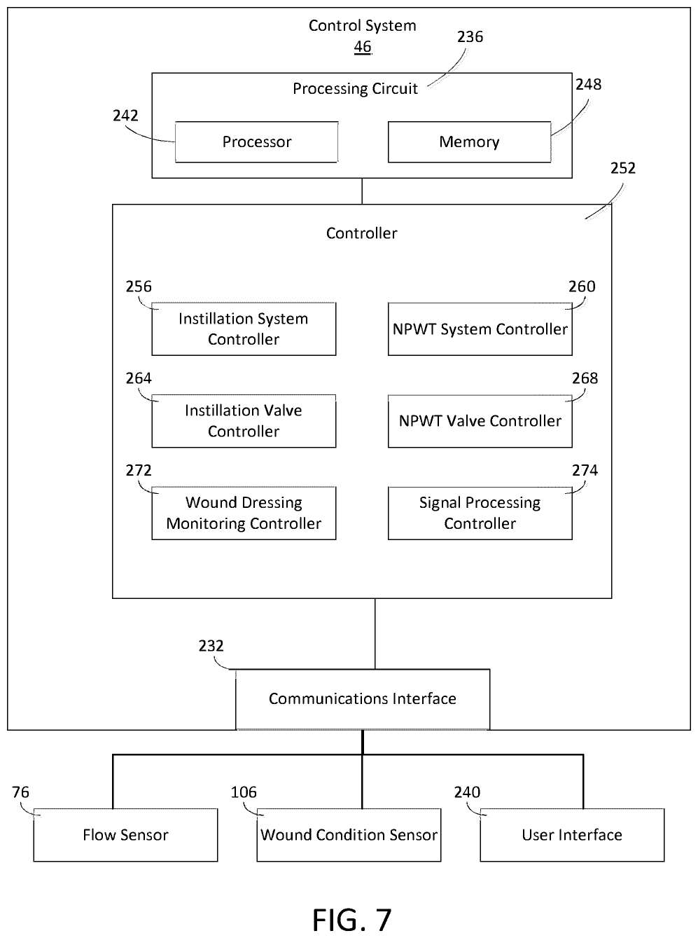

The instillation system 18 includes an instillation fluid source 70, an instillation fluid dispensing device 74, and a flow sensor 76 (FIG. 7). The instillation fluid source 70 stores an instillation fluid, such as a cleansing solution, a saline solution, a prescribed solution, an antibiotic, etc. The dispensing device 74 is connected to the instillation valve arrangement 26 of the valve arrangement 22 via the instillation conduit 78. The dispensing device 74 is structured to dispense the instillation fluid to the valve arrangement 22. The flow sensor 76 is engaged with the instillation system 18 to determine a flow rate of fluid dispensed by the instillation system 18. In some embodiments, the instillation fluid is added to the wound dressings 34, 38, 42 by the instillation system 18 and removed from the wound dressings 34, 38, 42 by the NPWT system 14. In such embodiments, the instillation fluid may remain in the wound dressing 34, 38, 42 for a predetermined period of time. The predetermined period of time in which the instillation fluid remains in the wound dressing 34, 38, 42 is generally referred herein to as a “dwell time.” In some embodiments, such as the embodiment shown in FIG. 1, the instillation system 18 may be integrally formed with the NPWT system 14. In such an embodiment, the negative pressure source 58 of the NPWT system 14 may generate negative pressure to dispense instillation fluid from the instillation fluid source 70 to the wound dressings 34, 38, 42.

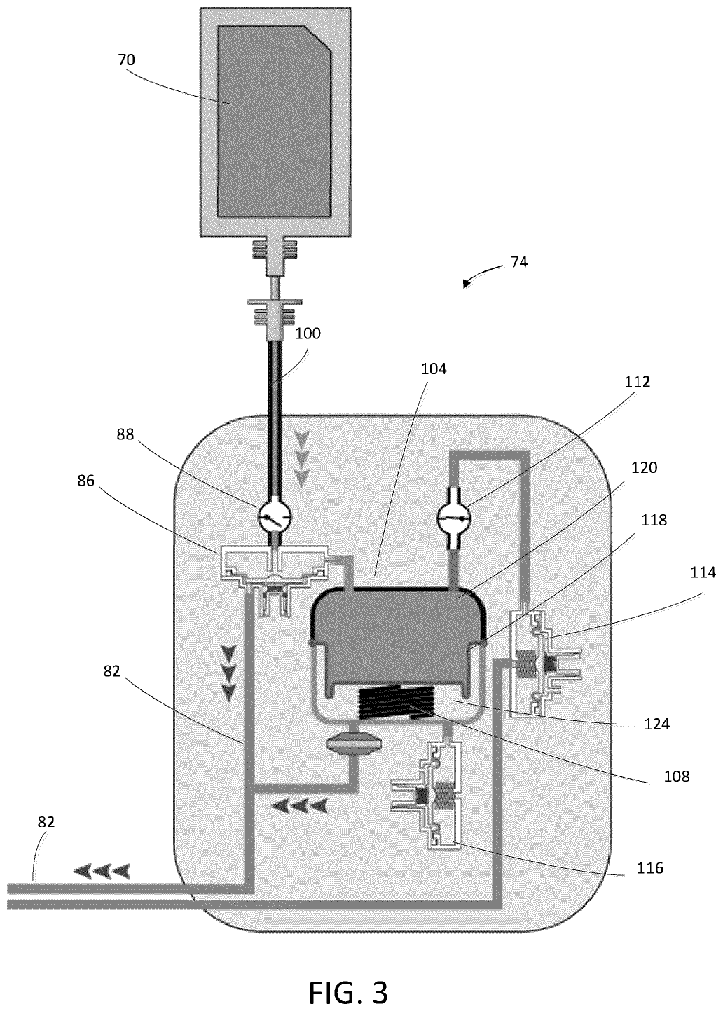

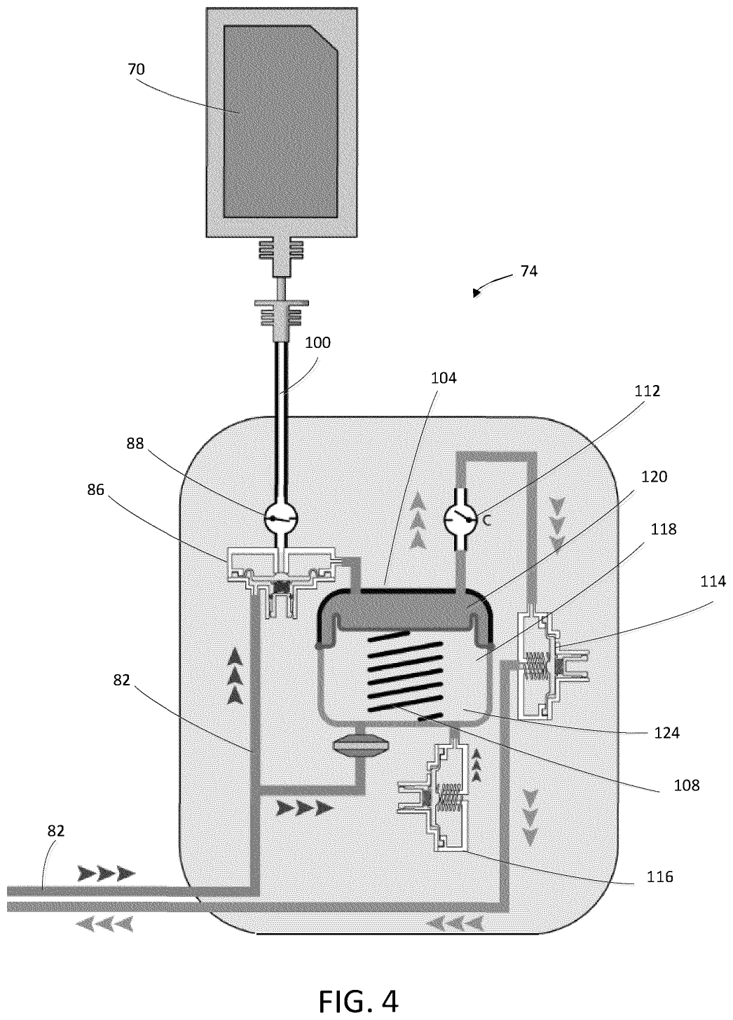

In some embodiments, such as the embodiment illustrated in FIG. 2, the instillation system 18 may be separate from the NPWT system 14. The dispensing device 74 may be a portable, single-use dispensing device 74. In such an embodiment, the dispensing device 74 may be a pneumatic dispensing device 74 that is powered by the negative pressure source 58. FIGS. 3-4 illustrate an exemplary pneumatic dispensing device 74 that is powered by negative pressure. The dispensing device 74 includes a pneumatic line 82, a pneumatic valve 86, a first check valve 88, a reservoir supply line 100, an instillation fluid reservoir 104, a compression spring 108, a second check valve 112, a dispensing valve 114, and an air supply valve 116. The instillation fluid reservoir 104 includes a movable diaphragm 118 and the compression spring 108. The diaphragm 118 divides the instillation fluid reservoir 104 into a fluid storage portion 120 and a spring portion 124.

The pneumatic valve 86 is positioned along the reservoir supply line 100 for allowing selective communication between the instillation fluid source 70 and the fluid storage portion 120 of the instillation fluid reservoir 104. The first check valve 88 is positioned along the reservoir supply line 100 for preventing backflow of fluid from the instillation fluid into the instillation fluid source 70. The pneumatic valve 86 is in fluid communication with the negative pressure source 58 and actuable by the negative pressure source 58. The dispensing valve 114 is positioned along the instillation conduit 78 for providing selective communication between the instillation fluid reservoir 104 and the instillation conduit 78. The second check valve 112 is positioned along the instillation conduit 78 for preventing backflow of the instillation fluid into the instillation fluid reservoir 104.

FIG. 3 illustrates the dispensing device 74 in a priming configuration. As illustrated by the arrows 130, the negative pressure source 58 generates negative pressure in the pneumatic line 82, which opens the pneumatic valve 86. Fluid flows from the instillation fluid source 70 through the instillation supply line 100 and the pneumatic valve 86 to the instillation fluid source 70 of the dispensing device 74. Since the dispensing valve 114 is closed and the first check valve 88 prevents backflow of fluid into the instillation fluid source 70 the diaphragm 118 is displaced downward against the bias of the compression spring 108 as the instillation fluid fills the instillation reservoir, compressing the compression spring 108 and generating positive pressure in the instillation fluid reservoir 104.

FIG. 4 illustrates the dispensing device 74 in an instillation configuration. As shown in FIG. 4, to dispense instillation fluid to one or more of the wound dressings 34, 38, 42, the dispensing valve 114 is opened. The positive pressure in the instillation reservoir 104 and the decompression of the compression spring 108 push the instillation fluid from the instillation fluid reservoir 104 into the instillation conduit 78 and into the one or more wound dressings 34, 38, 42. The second check valve 112 prevents backflow of instillation fluid into the instillation reservoir 104. The air supply valve 116 is open during instillation and allows air to flow into the spring portion 124 of the instillation fluid reservoir 104. In some embodiments, the dispensing device 74 may instead include a pump for pumping installation fluid into the wound dressings 34, 38, 42.

### Wound Dressings

By way of non-limiting example, as illustrated schematically in FIGS. 1-2, the wound therapy system 10 is shown to include the first wound dressing 34, the second wound dressing 38, and the third wound dressing 42. In other embodiments, the wound therapy system 10 may include more or fewer wound dressings. Each of the wound dressings 34, 38, 42 is dimensioned to treat a wound site of a patient. Accordingly, each of the wound dressings 34, 38, 42 may have similar or different dimensions (and define similar or different volumes) based on the size of the wound sites. The first wound dressing 34, the second wound dressing 38, and the third wound dressing 42 are substantially similar. Accordingly, only the first wound dressing 34 is described in detail below. Corresponding structures of the second wound dressing 38 are indicated using prime symbol “′”. Corresponding structures of the third wound dressing 42 are indicated using the double prime symbol “″.”

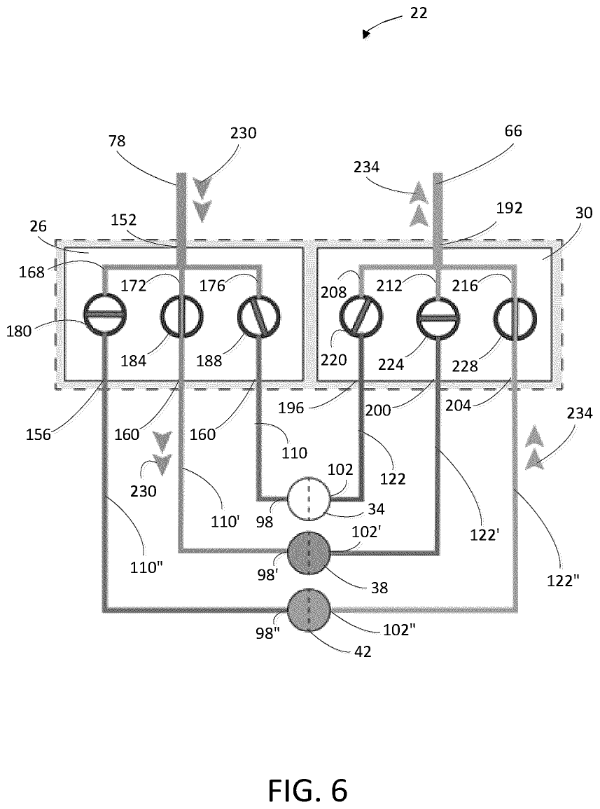

The first wound dressing 34 includes a drape (not shown), an absorbent layer (not shown), an instillation inlet 98 (FIG. 6), a NPWT outlet 102 (FIG. 6), a wound condition sensor 106 (FIG. 7), and a status indication system 54.

The drape is structured to overlie the absorbent layer and the wound site and administer a fluid-tight seal around the wound site. The drape supports the absorbent layer and administers a barrier to passage of microorganisms through the first wound dressing 34. This increases the total fluid handling capacity (TFHC) of the first wound dressing 34 while promoting a moist wound environment.

The absorbent layer is adapted to wick fluid (e.g. exudate) from the wound and includes in-molded manifold structures for distributing negative pressure throughout the first wound dressing 34 during negative pressure wound therapy treatments. The absorbent layer can be made from a porous and permeable foam-like material and, more particularly, a reticulated, open-cell polyurethane or polyether foam that allows good permeability of wound fluids while under a reduced pressure. In some embodiments, a non-adherent layer (not shown) may be positioned between the absorbent layer and the wound. The non-adherent layer is made of a material that is fluid-permeable and intended to not irritate the patient's skin.

The instillation inlet 98 is engaged with the drape and structured to receive an instillation conduit 110 that is in fluid communication with the instillation valve arrangement 26. In some embodiments, the instillation inlet 98 may include a single direction valve (e.g. check valve, etc.—not shown) to prevent instillation fluid from flowing backwards into the instillation conduit 110.

The NPWT outlet 102 is engaged with the drape and structured to receive a negative pressure conduit 122 that is in fluid communication with the NPWT valve arrangement 30. In some embodiments, the NPWT outlet 102 may include a single direction valve (not shown) to prevent instillation fluid and/or exudates from flowing backwards into the first wound dressing 34.

The wound condition sensor 106 is engaged with the first wound dressing 34 to sense information indicative of a condition of the first wound dressing 34. In some embodiments, the wound condition sensor 106 may be in wireless or wired communication with the control system 46. The wound condition sensor 106 is structured to sense information indicative of a fill rate of the first wound dressing 34. More specifically, the information indicative of the fill rate of the first wound dressing 34 can include an indication that the first wound dressing 34 is being filled gradually or rapidly. The wound condition sensor 106 is further structured to sense information indicating that the first wound dressing 34 is full. For example, in some embodiments, the wound condition sensor 106 may be a humidity or moisture sensor and the information indicative of the condition of the first wound dressing 34 may be a humidity of the first wound dressing 34. In such an embodiment, the control system 46 can determine whether the first wound dressing 34 is being filled gradually or rapidly based on the humidity (or rate of change of humidity) detected by the wound condition sensor 106. The control system 46 can further determine that the first wound dressing 34 is full based on the humidity sensed by the wound condition sensor 106. The control system 46 may be structured to control the instillation fluid source 70 and the instillation valve arrangement 26 of the valve arrangement 22 to administer instillation therapy to the first wound dressing 34 in response based on the information indicative of the state of the first wound dressing 34. In other embodiments, the wound condition sensor 106 may be a pressure sensor and the condition of the first wound dressing 34 can be a pressure of the first wound dressing 34. In another embodiment, the wound condition sensor 106 may be a pH sensor and the condition of the first wound dressing 34 can be a pH of the first wound dressing 34. In another embodiment, the wound condition sensor 106 may be a temperature sensor and the condition of the first wound dressing 34 can be a temperature of the first wound dressing 34. In another embodiment, the wound condition sensor 106 may be a capacitance sensor and the condition of the first wound dressing 34 can be a capacitance of the first wound dressing 34. The capacitance of the first wound dressing 34 can be indicative of a presence and/or an amount of fluid in the first wound dressing 34. In another embodiment, the wound condition sensor 106 may be a flow sensor and the condition of the first wound dressing 34 can be a flow rate of fluid into and/or out of the first wound dressing 34. In some embodiments, the NPWT system 14 may include sensors (not shown) structured to receive information indicative of the condition of the first wound dressing 34. Such sensors may function as backup sensors if the wound condition sensor 106 fails and/or such sensors may be used to confirm that the condition of the first wound dressing 34 is consistent. For example, in some embodiments, the NPWT system may include a flow sensor for sensing a flow rate of instillation into or out of the first wound dressing 34. In another embodiment, the NPWT system may include a pressure sensor for sensing a pressure generated by the negative pressure source 58.

The status indication system 54 is structured to indicate a status of the first wound dressing 34. In the illustrated embodiment, the status indication system 54 is shown to include a first status indicator 140 structured to indicate a first status, a second status indicator 144 structured to indicate a second status, and a third status indicator 148 structured to indicate a third status. By way of non-limiting example, in the illustrated embodiment, the first status indicator 140 is structured to indicate that the first wound dressing 34 is administering instillation therapy, the second status indicator 144 is structured to indicate that the first wound dressing 34 is administering NPWT, and the third status indicator 148 is structured to indicate that the first wound dressing 34 is neither administering NPWT nor instillation therapy. In the illustrated embodiment, the first status indicator 140, the second status indicator 144, and the third status indicator 148 are LEDs that are powered on to indicate the presence of the first status, the second status, or the third status, respectively. In other embodiments, the status indication system 54 may include colorimetric indicators, auditory indicators, and/or graphical or textual indicators displayed on a screen. In other embodiments, the status indication system 54 can include more or fewer status indicators. For example, the status indication system 54 can be structured to indicate additional and/or other wound dressing statuses, such as a blockage or a leak in the first wound dressing 34.

Table 1 shows wound dressing statuses that can be indicated by the status indication system 54 for wound dressings 34, 38, 42 that are undergoing NPWT therapy. As is shown in greater detail below, the status indication system 54 can indicate a blockage in tubing extending between the NPWT system 14 and the wound dressings 34, 38, 42, a fluid leak in the wound dressing 34, 38, 42, an air leak in the wound dressing 34, 38, 42, and/or desiccation (e.g., dryness) in the wound dressing 34, 38, 42. In some embodiments, the wound dressing statuses can be determined based on information indicative of the condition of the first wound dressing 34 sensed by the wound condition sensor 106. For example, the wound dressing statuses can be based on a sensed wound dressing pressure, a sensed wound dressing humidity, and/or a sensed wound dressing temperature. In other embodiments, the wound dressing statuses can be determined based on information related to the NPWT system 14. For example, in some embodiments, the wound dressing statuses shown in Table 1 can be determined based on a duty cycle of a pump of the NPWT system 14 and/or a pump pressure of the NPWT system 14. The duty cycle of the pump is an indication of how often the pump runs for a given time period. For example, the duty cycle can be a ratio of the pump run time to the total length of the given time period.

TABLE 1
Negative Pressure State Indication

| Inputs | Tubing Blockage | Fluid Leak | Air Leak | Desiccation |
|---|---|---|---|---|
| Sensed Wound | WP may slowly decrease | WP decreases | WP may decrease | WP likely unaffected |
| Pressure (WP) | | | | |
| Sensed Wound | Wound dressing humidity | Wound dressing | Wound dressing humidity | Wound dressing |
| Dressing Humidity | may slowly increase | humidity increases | can change rapidly | humidity increases |
| Sensed Wound | Temperature may slowly increase | Temperature may increase | Temperature may change | Temperature may change |
| Dressing Temperature | | | | |
| NPWT Pump System | PD increases | PD may increase | PD increases | PD likely unaffected |
| Duty Cycle (PD) | | | | |
| NPWT Pump System | PP increases | PP can increase for a short | PP can increase | PP likely unaffected |
| Pressure (PP) | | time and then decrease | | |

Table 2 shows wound dressing statuses that can be indicated by the status indication system 54 for wound dressings 34, 38, 42 that are undergoing instillation therapy. As is shown in greater detail below, the status indication system 54 can indicate a blockage in tubing extending between the instillation system 18 and the wound dressings 34, 38, 42, a fluid leak in the wound dressing 34, 38, 42, and/or a fill status of the wound dressing 34, 38, 42. For example, the wound dressing statuses can be based on a sensed wound dressing pressure, a sensed wound dressing humidity, and/or a sensed wound dressing temperature. In other embodiments, the wound dressing statuses can be determined based on information related to the instillation system 18. For example, in embodiments in embodiments in which the instillation system 18 includes a pump, the wound dressing statuses shown in Table 2 can be determined based on a duty cycle of a pump of the instillation system and/or a pump pressure of the instillation system 18. The duty cycle of the pump is an indication of how often the pump runs for a given time period. For example, the duty cycle can be a ratio of the pump run time to the total length of the given time period. In some embodiments, the instillation system 18 may include an occlusion sensor for sensing a blockage within the instillation system 18 (e.g. within the tubing described with respect to the dispensing deice 74).

TABLE 2
Fluid Instillation State Indication

| Inputs | Tubing Blockage | Dressing Fluid Leaks | Fill Status |
|---|---|---|---|
| Sensed Wound | WP does not change | WP does not change | WP may increase |
| Pressure (WP) | | | |
| Sensed Wound | Wound dressing humidity | Wound dressing humidity | Wound dressing |
| Dressing Humidity | does not increase | may not increase | humidity increases |
| Sensed Wound | Wound dressing temperature | Wound dressing | Wound dressing |
| Dressing Temperature | does not decrease | temperature may increase | temperature decreases |
| Instillation Pump | IP increases | IP may increase | N/A |
| System Duty Cycle (ID) | | | |
| Instillation Pump | IP increases | IP may increase | N/A |
| System Pressure (IP) | | | |
| Sensed Occlusion | May trip | N/A | N/A |
| (Pump System) | | | |

In some embodiments, at least one of the wound dressings 34, 38, 42 may be a pair of separate (e.g, fluidly separated) wound dressings. In such an embodiment, one of the paired wound dressings is an instillation wound dressing and one of the paired wound dressings is a NPWT wound dressing. The instillation wound dressing includes an instillation inlet substantially similar to the instillation inlet 98, a wound condition sensor substantially similar to the wound condition sensor 106, and a status indication system substantially similar to the status indication system 54. The instillation inlet is structured to receive an instillation conduit that is in fluid communication with the instillation valve arrangement 26. The NPWT wound dressing includes a NPWT outlet substantially similar to the NPWT outlet 102, a wound condition sensor substantially similar to the wound condition sensor 106, and a status indication system substantially similar to the status indication system 54. The NPWT outlet is structured to receive a negative pressure conduit that is in fluid communication with the NPWT valve arrangement 30. The paired wound dressing can be used to treat particularly large wounds.

### Valve Arrangement

As shown in FIGS. 3-4, the valve arrangement 22 includes an instillation valve arrangement 26 and a NPWT valve arrangement 30. The instillation valve arrangement 26 is in fluid communication with the instillation system 18 and the wound dressings 34, 38, 42 to selectively administer instillation therapy through at least one of wound dressings 34, 38, 42.

The NPWT valve arrangement 30 is in fluid communication with the NPWT system 14 and the wound dressings 34, 38, 42 to selectively administer NPWT through at least one of the wound dressings 34, 38, 42. In the illustrated construction, the valve arrangement 22 (e.g. both the instillation valve arrangement 26 and the NPWT valve arrangement 30) is integrally formed with the NPWT system 14. In other embodiments, the instillation valve arrangement 26 may be structurally separate from the NPWT valve arrangement 30. In such embodiments, the instillation valve arrangement 26 may be positioned at any point along the instillation conduit 78 and/or the instillation conduits 110, 110′, 110″. For example, the instillation valve arrangement 26 may be positioned proximate a fluid flow splitter and/or a Y-connection connecting the wound dressings 34, 38, 42 to the instillation fluid source 70. In embodiments in which the instillation valve arrangement 26 is structurally separate from the NPWT valve arrangement 30, the instillation valve arrangement 26 includes a power supply (not shown) and a wireless communication interface (not shown).

As shown in FIGS. 3-4, the instillation valve arrangement 26 includes an instillation source port 152, a first instillation wound dressing port 156, a second instillation wound dressing port 160, and a third instillation wound dressing port 164. The instillation source port 152 is structured to engage the instillation conduit 78 of the instillation system 18. The first instillation wound dressing port 156, the second instillation wound dressing port 160, and the third instillation wound dressing port 164 are structured for fluid communication with the first wound dressing 34, the second wound dressing 38, and the third wound dressing 42, respectively. For example, in the illustrated embodiment, the first instillation wound dressing port 156 is engaged with the instillation conduit 110, the second instillation wound dressing port 160 is engaged with the instillation conduit 110′, and the third wound dressing port is engaged with the instillation conduit 110″.

With continued reference to FIGS. 3-4, the instillation valve arrangement 26 further includes a first instillation flow path 168, a second instillation flow path 172, and a third instillation flow path 176. The first instillation flow path 168 is defined between the instillation source port 152 and the first instillation wound dressing port 156. A first instillation valve 180 is positioned along the first instillation flow path 168. The first instillation valve 180 is positionable to allow fluid communication between the instillation system 18 and the first wound dressing 34 or to prevent fluid communication between the instillation system 18 and the first wound dressing 34. The second instillation flow path 172 is defined between the instillation source port 152 and the second instillation wound dressing port 160. A second instillation valve 184 is positioned along the second instillation flow path 172. The second instillation valve 184 is positionable to allow fluid communication between the instillation system 18 and the second wound dressing 38 or prevent fluid communication between the instillation system 18 and the second wound dressing 38. The third instillation flow path 176 is defined between the instillation source port 152 and the third instillation wound dressing port 164. A third instillation valve 188 is positioned along the third instillation flow path 176. The third instillation valve 188 is positionable to allow fluid communication between the instillation system 18 and the third wound dressing 42. The first instillation valve 180, the second instillation valve 184, and the third instillation valve 188 can be controlled independently of each other. Accordingly, it is possible to administer instillation fluid to the first wound dressing 34, the second wound dressing 38, and the third wound dressing 42 at different times and/or for different amounts of time by independently positioning the first instillation valve 180, the second instillation valve 184, and third instillation valve 188, respectively. It is also possible to simultaneously administer instillation fluid to the first wound dressing 34, the second wound dressing 38, and the third wound dressing 42 at different flow rates based on the positions of the first instillation valve 180, the second instillation valve 184, and the third instillation valve 188, respectively.

As shown in FIG. 3, in some embodiments, the instillation valves 180, 184, 188, are solenoid valves and are repositionable between an open position in which the instillation valves 180, 184, 188 are positioned to allow instillation fluid to flow along the first instillation flow path 168, the second instillation flow path 172, and the third instillation flow path 176, respectively, and a closed position, in which the instillation valves 180, 184, 188 are positioned to prevent fluid from flowing along the first instillation flow path 168, the second instillation flow path 172, and the third instillation flow path 176, respectively.

As shown in FIG. 6, in some embodiments, the instillation valves 180, 184, 188, are continuously repositionable between the open position and the closed position. Accordingly, the instillation valves 180, 184, 188, can throttle the flow of instillation fluid between along the first instillation flow path 168, the second instillation flow path 172, and the third instillation flow path 176. In some embodiments, the instillation valves 180, 184, 188 are controlled by the control system 46, as is described in greater detail below. In other embodiments, the instillation valves 180, 184, 188 can be manually controlled.

In some embodiments, the instillation valve arrangement 26 may be integrally formed with the wound dressings 34, 38, 42. In such embodiments, the instillation valves 180, 184, 188 are structured for wired and/or wireless communication with the control system 46. The instillation valves 180, 184, 188 each further include a power source (not shown), such as a battery, for energizing the instillation valves 180, 184, 188. In embodiments in which only instillation or NPWT will be applied to the wound dressings, the valve arrangement 22 may include only the instillation valve arrangement 26 or the NPWT valve arrangement 30, respectively.

The NPWT valve arrangement 30 is structured similarly to the instillation valve arrangement 26. As shown in FIGS. 3-4, the NPWT valve arrangement 30 includes an NPWT source port 192, a first NPWT wound dressing port 196, a second NPWT wound dressing port 200, and a third NPWT wound dressing port 204. The NPWT source port 192 is structured to engage the negative pressure conduit 66 of the NPWT system 14. The first NPWT wound dressing port 196, the second NPWT wound dressing port 200, and the third NPWT wound dressing port 204 are structured for fluid communication with the first wound dressing 34, the second wound dressing 38, and the third wound dressing 42, respectively. For example, in the illustrated embodiment, the first NPWT wound dressing port 196 is engaged with the negative pressure conduit 122, the second NPWT wound dressing port 200 is engaged with negative pressure conduit 122′, and the third NPWT wound dressing port 204 is engaged with the negative pressure conduit 122″.

With continued reference to FIGS. 3-4, the NPWT valve arrangement 30 further includes a first negative pressure flow path 208, a second negative pressure flow path 212, and a third negative pressure flow path 216. The first negative pressure flow path 208 is defined between the NPWT source port 192 and the first NPWT wound dressing port 196. A first negative pressure valve 220 is positioned along the first negative pressure flow path 208. The first negative pressure valve 220 is positionable to allow fluid communication between the NPWT system 14 and the first wound dressing 34 or to prevent fluid communication between the NPWT system 14 and the first wound dressing 34. The second negative pressure flow path 212 is defined between the NPWT source port 192 and the second NPWT wound dressing port 200. A second negative pressure valve 224 is positioned along the second negative pressure flow path 212. The second negative pressure valve 224 is positionable to allow fluid communication between the NPWT system 14 and the second wound dressing 38 or prevent fluid communication between the NPWT system 14 and the second wound dressing 38. The third negative pressure flow path 216 is defined between the NPWT source port 192 and the third NPWT wound dressing port 136. A third negative pressure valve 228 is positioned along the third negative pressure flow path 216. The third negative pressure valve 228 is positionable to allow fluid communication between the NPWT system 14 and the third wound dressing 42.

The first negative pressure valve 220, the second negative pressure valve 224, and the third negative pressure valve 228 can be controlled independently of each other. Accordingly, it is possible to administer negative pressure to the first wound dressing 34, the second wound dressing 38, and the third wound dressing 42 at different times and/or for different amounts of time by independently positioning the first negative pressure valve 220, the second negative pressure valve 224, and third negative pressure valve 228, respectively. It is also possible to simultaneously administer negative pressure to the first wound dressing 34, the second wound dressing 38, and the third wound dressing 42 at different negative pressures based on the positions of the first negative pressure valve 220, the second negative pressure valve 224, and the third negative pressure valve 228, respectively.

As shown in FIG. 3, in some embodiments, the negative pressure valves 220, 224, 228, are solenoid valves and are repositionable between an open position in which the negative pressure valves 220, 224, 228 are positioned to allow the negative pressure source 58 to administer negative pressure via the first negative pressure flow path 208, the second negative pressure flow path 212, and the third negative pressure flow path 216, respectively, and a closed position, in which the negative pressure valves 220, 224, 228 prevent the negative pressure source 58 from providing negative pressure via the first negative pressure flow path 208, the second negative pressure flow path 212, and the third negative pressure flow path 216, respectively.

As shown in FIG. 6, in some embodiments, the negative pressure valves 220, 224, 228, are continuously repositionable between the open position and the closed position. Accordingly, the negative pressure valves 220, 224, 228, can throttle the suction between along the first negative pressure flow path 208, the second negative pressure flow path 212, and the third negative pressure flow path 216.

FIG. 6 illustrates a detail view of the valve arrangement 22 engaged with the first wound dressing 34, the second wound dressing 38, and the third wound dressing 42. The instillation inlet 98 of the first wound dressing 34 is connected to the first instillation wound dressing port 156 via the instillation conduit 110. The NPWT outlet 102 of the first wound dressing 34 is connected to the first NPWT wound dressing port 196 via the negative pressure conduit 122. Both the first instillation valve 180 and the first negative pressure valve 220 are positioned a in transitional position between the fully open position and the fully closed position. Accordingly, the first instillation valve 180 and the first negative pressure valve 220 are throttling fluid flow. Since the first instillation valve 180 and the first negative pressure valve 220 are in transitional positions, instillation fluid is simultaneously entering and leaving the first wound dressing 34. The instillation inlet 98′ of the second wound dressing 38 is connected to the second instillation wound dressing port 160 via the instillation conduit 110′. The NPWT outlet 102′ of the second wound dressing 38 is connected to the second NPWT wound dressing port 200 via the negative pressure conduit 122′. The second instillation valve 184 is positioned in the fully open position. The second negative pressure valve 224 is positioned in the fully closed position. Accordingly, as indicated by the arrows 230, the second wound dressing 38 is receiving instillation fluid and is not in fluid communication with the NPWT system 14. The instillation inlet 98″ of the third wound dressing 42 is connected to the third instillation wound dressing port 164 via the instillation conduit 110″. The NPWT outlet 102″ of the third wound dressing 42 is connected to the third negative pressure wound dressing port 204 via the negative pressure conduit 122″. The third instillation valve 188 is positioned in the fully closed position. The third negative pressure valve 228 is positioned in the fully open position. Accordingly, as indicated by the arrows 234, the third wound dressing 42 is receiving negative pressure and is not in fluid communication with the instillation system 18. Accordingly, the valve arrangement 22 allows the wound dressings 34, 38, 42 to simultaneously administer a combination of instillation and NPWT, instillation therapy only, and NPWT only, respectively, from the single instillation source 18 and the single NPWT source 14.

Accordingly, as shown in FIG. 6, the first instillation valve 180 and the first negative pressure valve 220 are positionable to allow fluid to flow through the first instillation flow path 168 (e.g. the first instillation valve 180 is in one of the open or transitional positions and the first negative pressure valve 220 is in the closed position), the first instillation valve 180 and the first negative pressure valve 220 are positionable to allow fluid to flow through the first negative pressure flow path 208 (e.g. the first instillation valve 180 is in the closed position and the first negative pressure valve 220 is one of the open or transitional positions), or the first instillation valve 180 and the first negative pressure valve 220 are positionable to allow fluid to flow through both the first instillation flow path 168 and the first negative pressure flow path 208 (e.g., both the first instillation valve 180 and the first negative pressure valve 220 are in one of the open position or the transitional position). The second instillation valve 184 and the second negative pressure valve 224 can be positioned in a similar manner to the first instillation valve 180 and the first negative pressure valve 220. Likewise, the third instillation valve 188 and the third negative pressure valve 228 can be positioned in a similar manner to the first instillation valve 180 and the first negative pressure valve 220.

### Controller

Referring now to FIG. 7, a block diagram illustrating the control system 46 in greater detail is shown, according to an exemplary embodiment. The control system 46 is shown to include a communications interface 232, a processing circuit 236, and a user interface 240. The communications interface 232 may facilitate communications between the control system 46 and external systems or devices. For example, the communications interface 232 may receive information indicative of the flow rate through the instillation valve arrangement 26 from the flow sensor 76. In some embodiments, the communications interface 232 receives information indicative of the condition of the wound dressings 34, 38, 42, from the wound condition sensors 106, 106′, 106″. In some embodiments, the wound condition sensors 106, 106′, 106″ may be humidity sensors and the information indicative of the condition of the wound dressings 34, 38, 42 may be a humidity of the wound dressings 34, 38, 42. In other embodiments, the wound condition sensors 106, 106′, 106″ may be pressure sensors and the information indicative of the condition of the wound dressings 34, 38, 42 may be a pressure of the wound dressings 34, 38, 42. In another embodiment, the wound condition sensors 106, 106′, 106″ may be a pH sensor and the condition of the wound dressings 34, 38, 42 can be a pH of the wound dressings 34, 38, 42. In another embodiment, the wound condition sensor 106 may be a temperature sensor and the condition of the wound dressings 34, 38, 42 can be a temperature of the wound dressings 34, 38, 42. The communications interface 232 can be structured to send control signals to the NPWT system 14, the instillation system 18, the pneumatic valve 86 of the dispensing device 74, the instillation valves 180, 184, 188 of the instillation valve arrangement 26 of the valve arrangement 22, and the negative pressure valves 220, 224, 228 of the NPWT valve arrangement 30 of the valve arrangement 22.

The communications interface 232 may include wired or wireless communications interfaces (e.g., jacks, antennas, transmitters, receivers, transceivers, wire terminals, etc.) for conducting data communications external systems or devices. In various embodiments, the communications may be direct (e.g., local wired or wireless communications) or via a communications network (e.g., a WAN, the Internet, a cellular network, etc.). For example, the communications interface 232 can include an Ethernet card and port for sending and receiving data via an Ethernet-based communications link or network. In another example, the communications interface 232 can include a Wi-Fi transceiver for communicating via a wireless communications network or cellular or mobile phone communications transceivers.

The processing circuit 236 is shown to include a processor 244 and memory 248. The processor 244 may be a general purpose or specific purpose processor, an application specific integrated circuit (ASIC), one or more field programmable gate arrays (FPGAs), a group of processing components, or other suitable processing components. The processor 244 is structured to execute computer code or instructions stored in memory 248 or received from other computer readable media (e.g., CDROM, network storage, a remote server, etc.).

The user interface 240 is structured to receive information indicative of the treatment parameters for the wound therapy system 10 input by an operator of the wound therapy system 10. The treatment parameters may include a number of wound dressings to be used with the wound therapy system 10. The treatment parameters may include a type of therapy for each of the wound dressings 34, 38, 42. For example, the operator may indicate whether each of the wound dressings is administering instillation therapy, NPWT, and/or a combination of both instillation therapy and NPWT. In embodiments in which the wound dressing is to administer NPWT, the treatment parameters may include a negative pressure for the NPWT. In embodiments in which the wound dressings are to administer instillation therapy and/or a combination of instillation therapy and NPWT, the treatment parameters may include a dwell time for the instillation therapy and/or the NPWT. In some embodiments, the treatment parameters input by the operator via the user interface 240 may be saved in the memory 248.

The memory 248 may include one or more devices (e.g., memory units, memory devices, storage devices, etc.) for storing data and/or computer code for completing and/or facilitating the various processes described in the present disclosure. The memory 248 may include random access memory (RAM), read-only memory (ROM), hard drive storage, temporary storage, non-volatile memory, flash memory, optical memory, or any other suitable memory for storing software objects and/or computer instructions. The memory 248 may include database components, object code components, script components, or any other type of information structure for supporting the various activities and information structures described in the present disclosure. The memory 248 may be communicably connected to processor 244 via processing circuit 236 and may include computer code for executing (e.g., by processor 244) one or more processes described herein. When the processor 244 executes instructions stored in the memory 248, the processor 244 generally configures the control system 46 (and more particularly processing circuit 236) to complete such activities.

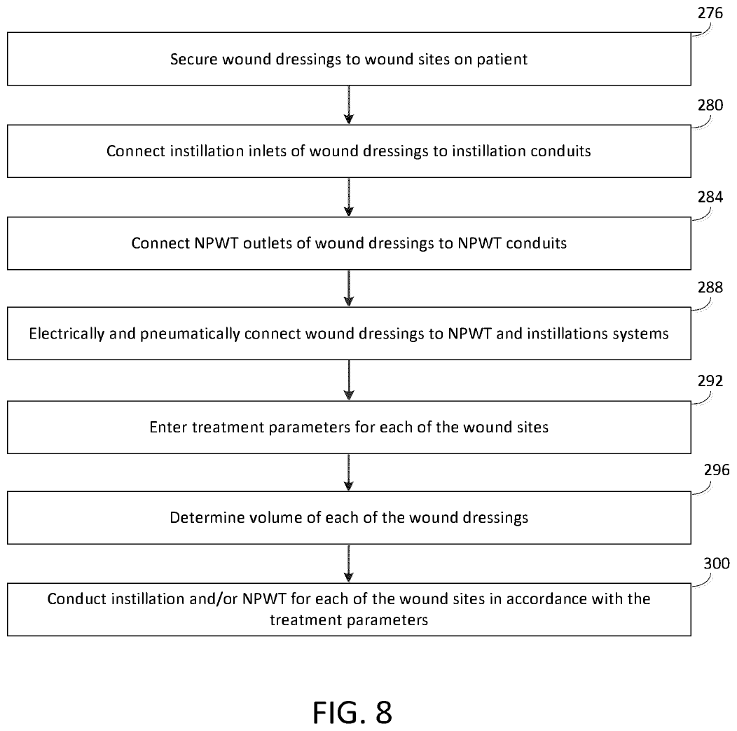

The memory 248 is shown to include a controller 252. The controller 252 may include various functional modules, shown as separate components in FIG. 8. For example, the controller 252 is shown to include an instillation system controller 256, a NPWT system controller 260, an instillation valve controller 264, negative pressure valve controller 268, a wound dressing monitoring controller 272, and a signal processing controller 274.

The instillation system controller 256 is structured to control the instillation system 18. For example, the instillation system controller 256 can control a flow rate of the instillation fluid dispensed from the instillation fluid source 70 through the dispensing device 74 to the instillation valve arrangement 26 of the valve arrangement 22. In embodiments in which the dispensing device 74 is the pneumatic device including the pneumatic valve 86, the instillation system controller 256 can control a position of the pneumatic valve 86. In embodiments in which the dispensing device 74 is a pump, the instillation system controller 256 can control the pump.

The NPWT system controller 260 is structured to control the NPWT system 14. For example, the NPWT system controller 260 is structured to send control signals to the negative pressure source 58 to control a suction force generated by the negative pressure source 58.

The instillation valve controller 264 is structured to control the positions of the instillation valves 180, 184, 188 based on the treatment parameters received via the user interface 240. The instillation valve controller 264 can control the instillation valves 180, 184, 188 independently of each other. Accordingly, the instillation valve controller 264 can position the instillation valves 180, 184, 188 of the wound dressings 34, 38, 42 administering instillation therapy to allow a predetermined amount of instillation fluid to the wound dressings 34, 38, 42, while positioning the instillation valves 180, 184, 188 of the wound dressings 34, 38, 42 not administering instillation therapy in the closed position. In embodiments in which the instillation valves 180, 184, 188 are solenoid valves, the instillation valve controller 264 can control an amount of time that the instillation valves 180, 184, 188 are opened to administer the desired amount of instillation fluid to the wound dressings 34, 38, 42, respectively. In embodiments in which the instillation valves 180, 184, 188 are continuously repositionable between the open and closed positions (e.g. the instillation valves 180, 184, 188 can throttle the fluid flow through the first instillation flow path 168, the second instillation flow path 172, and the third instillation flow path 176, respectively), the instillation valve controller 264 can determine a position of each of the instillation valves 180, 184, 188 to administer the desired amount of instillation fluid to the wound dressings 34, 38, 42.

In some embodiments, the treatment parameters may allow the user to determine the timing of instillation therapy and/or NPWT for each of the wound dressings 34, 38, 42. For example, the user can set the instillation system 18 to begin filling each of the wound dressings 34, 38, 42 with instillation fluid at the same time. In such an embodiment, the instillation valve controller 264 determines when to close each of the instillation valves 180, 184, 188. In embodiments in which the instillation valves 180, 184, 188 are structured to throttle the instillation fluid flow, the instillation valve controller 264 determines a position of each of the instillation valves 180, 184, 188. The instillation valve controller determines when to close the instillation valves 180, 184, 188 and/or a position of the instillation valves 180, 184, 188 based on the volume of the wound dressings 34, 38, 42 and the flow rate of instillation fluid through the dispensing device 74. In other embodiments, the operator can specify that different wound dressings 34, 38, 42 receive instillation fluid at different times. In such an embodiment, the instillation valve controller 264 is structured to command the instillation valves 180, 184, 188 to open and close in accordance with the different instillation times. Such a flow regime may be used when at least one of the wound sites has a higher risk of drying out than the other wound sites being treated.

The instillation system controller 256 and the instillation valve controller 264 are structured to cooperatively determine a volume of each of the wound dressings 34, 38, 42. For example, during the first instillation treatment, the instillation system controller 256 and the instillation valve controller 264 are structured to determine a volume of each of the wound dressings 34, 38, 42. The instillation valve controller 264 opens the instillation valves 180, 184, 188 individually in series so that the instillation system controller 256 can fill the wound dressings 34, 38, 42. The instillation system controller 256 determines a volume of instillation fluid added to each of the wound dressings 34, 38, 42 and stores the volume of each of the wound dressings 34, 38, 42 in the memory 248. In some embodiments, the instillation controller can determine the volume of each of the wound dressings 34, 38, 42 based on the flow rate of the instillation fluid dispensed by the dispensing device 74 and sensed by the flow sensor 76 and an amount of time that elapsed while the wound dressing 34, 38, 42 was being filled. During subsequent instillation steps, the instillation valve controller 264 can then determine how much instillation fluid to dispense to the wound dressings 34, 38, 42 based on the volumes determined for each of the wound dressings 34, 38, 42. In other embodiments, the instillation system controller 256 can command the dispensing device 74 to dispense instillation fluid into the wound dressings 34, 38, 42 until the information indicative of the wound condition sensed by the wound condition sensors 106, 106′, 106″ indicates that the wound dressings 34, 38, 42 are full. When the wound dressings 34, 38, 42 are full, the instillation system controller 256 can command the instillation valves 180, 184, 188 to close. Accordingly, in some embodiments, the flow rate of instillation fluid into the wound dressings 34, 38, 42 can be determined based on the volume of the wound dressings 34, 38, 42. In some embodiments, the instillation valves 180, 184, 188 and the negative pressure valves 220, 224, 228 can be positioned based on the condition of the wound dressings 34, 38, 42.

The negative pressure valve controller 268 is structured to control the positions of the negative pressure valves 220, 224, 228 based on the treatment parameters received via the user interface 240. The negative pressure valve controller 268 can control the negative pressure valves 220, 224, 228 independently of each other. Accordingly, the negative pressure valve controller 268 can position the negative pressure valves 220, 224, 228 of the wound dressings 34, 38, 42 administering NPWT to subject the wound dressings 34, 38, 42 to a predetermined amount of negative pressure, while positioning the negative pressure valves 220, 224, 228 of the wound dressings 34, 38, 42 not administering NPWT in the closed position. In embodiments in which the negative pressure valves 220, 224, 228 are solenoid valves, the negative pressure valve controller 268 can control an amount of time that the negative pressure valves 220, 224, 228 are opened to administer the desired amount of negative pressure to the wound dressings 34, 38, 42, respectively. In embodiments in which the negative pressure valves 220, 224, 228 are continuously repositionable between the open and closed positions (e.g. the negative pressure valves 220, 224, 228 can throttle the fluid flow through the first negative pressure flow path 208, the second negative pressure flow path 212, and the third negative pressure flow path 216, respectively), the negative pressure valve controller 268 can determine a position of each of the negative pressure valves 220, 224, 228 to administer the desired amount of negative pressure to the wound dressings 34, 38, 42.

In embodiments in which at least two of the wound dressings 34, 38, 42 are to administer an instillation therapy regime in which instillation fluid is to remain in the wound dressing for a predetermined dwell time, the negative pressure valve controller 268 may start a timer in response to receiving information indicating that the wound dressings 34, 38, 42 are filled from the wound condition sensors 106, 106′, 106″. In response to determining that the dwell time has elapsed, the negative pressure valve controller can command the negative pressure valves 220, 224, 228 to open to drain the instillation fluid from the wound dressings 34, 38, 42 to the collection chamber. In response to determining, based on information from the wound condition sensors 106, 106′, 106″, that the instillation fluid has drained from wound dressings 34, 38, 42, the negative pressure valve controller can close the negative pressure valves 220, 224, 228 so that the wound dressings 34, 38, 42 can be refilled. In embodiments in which the wound dressings 34, 38, 42 administer a combination of instillation therapy and NPWT, the negative pressure valves 220, 224, 228 may be left open and/or repositioned to subject the wound dressings 34, 38, 42 to the predetermined amount of negative pressure.

The wound dressing monitoring controller 272 is structured to monitor the conditions of the wound dressings 34, 38, 42 based on the information indicative of the wound conditions sensed by the wound condition sensors 106, 106′, 106″. The wound dressing monitoring controller 272 is structured to determine a state of the wound dressings 34, 38, 42 based on the information indicative of the wound conditions sensed by the wound condition sensors 106, 106′, 106″. In response to determining the status of the wound dressings 34, 38, 42, the wound dressing monitoring controller 272 is structured to command the status indication system 54 to indicate the status of each of the wound dressings 34, 38, 42. For example, in the embodiment illustrated in FIGS. 1-2, the wound dressing monitoring controller 272 commands the status indication system 54 to illuminate the status indicator 140, 144, 148, corresponding to the status of each of the wound dressings 34, 38, 42, respectively. In some embodiments, the wound dressing monitoring controller 272 determines the status of the each of the wound dressings 34, 38, 42 based on the information indicative of the wound conditions sensed by the wound condition sensors 106, 106′, 106″. In other embodiments, the wound dressing monitoring controller 272 may determine the status of each of the wound dressings 34, 38, 42 based at least in part on the positions of the instillation valves 180, 184, 188 and/or the negative pressure valves 220, 224, 228.

The signal processing controller 274 is structured to use distance sensing to prevent communications signals sent by nearby wound therapy systems 10 from interacting (e.g., to prevent electronic communication between nearby wound therapy systems 10). For example, the signal processing controller 274 may determine a distance between at least three components of the wound therapy system 10 (e.g. the NPWT system 14 and at least two of the wound dressings 34, 38, 42) based on a speed of communication signals and an amount of time elapsed between the time the communication signal is sent by one of the components of the wound therapy system 10 and the time that the communication signal is received by another of the components of the wound therapy system 10. The components of the wound therapy system 10 wound then ignore communication signals originating farther away than the distance or a predetermined distance based on the determined distance. In some embodiments, the memory 248 can include a position database that includes predetermined distance values that indicate, based on a distance between at least one of the NPWT system 14 and the instillation system 18 and the wound dressings 34, 38, 42 whether the NPWT system 14 and/or the instillation system 18 can control the wound dressings 34, 38, 42. In some embodiments, the position database includes predetermined distance values that indicate, based on relative positions between different ones of the wound dressings 34, 38, 42, whether the wound dressings 34, 38, 42 can be treating the same patient.

In some embodiments, one or more of the wound dressings 34, 38, 42 can compare the distance between other nearby wound dressings and the one or more wound dressings 34, 38, 42 and or the signal processing controller 274 can verify that the other nearby wound dressings within a predetermined distance of the wound dressings 34, 38, 42 are in electronic communication with the same NPWT system 14 and/or the same instillation system 18. In some embodiments, one or more of the wound dressings 34, 38, 42 can determine the relative positions between the one or more wound dressings 34, 38, 42 and other nearby wound dressings. The one or more wound dressings 34, 38, 42 and/or the signal processing controller 274 can determine, based on the determined relative positions, whether the other nearby wound dressings are likely treating the same patient as the one or more wound dressings 34, 38, 42. The wound dressings 34, 38, 42 and/or the signal processing controller 274 can then verify that the wound dressings 34, 38, 42 determined to be treating the same patient are in electronic communication with the same NPWT system 14 and/or the same instillation system 18. The wound dressings 34, 38, 42 and/or the signal processing controller 274 can also verify that the wound dressings determined not to be treating the same patient as the wound dressings 34, 38, 42 are not in electronic communication with the same NPWT system 14 and/or instillation system 18 as the wound dressings 34, 38, 42.

In some embodiments, the signal processing controller 274 can be structured to generate a binding signal to bind the NPWT system 14 with the wound dressings 34, 38, 42 to provide electronic communication between the NPWT system 14. The signal processing controller 274 can be structured to generate a release signal to prevent electronic communication between the NPWT system 14 and the wound dressings 34, 38, 42. After the wound dressings 34, 38, 42 have been paired with the NPWT system 14, the wound dressings 34, 38, 42 cannot be paired with another NPWT system until the wound dressings have been released from the NPWT system 14. In embodiments in which the instillation system 18 is a separate system from the NPWT system 14, the signal processing controller 274 can bind the instillation system 18 to the wound dressings 34, 38, 42 and release the instillation system 18 from the wound dressings 34, 38, 42 as described with respect to the NPWT system 14 and the wound dressings 34, 38, 42.

Although the instillation valves 180, 184, 188 and the negative pressure valves 220, 224, 228 are described as being positioned by the control system 46, in some embodiments, the instillation valves 180, 184, 188 and the negative pressure valves 220, 224, 228 can be positioned manually.

### Operation

FIG. 8 illustrates a flowchart of a method of use of the wound therapy system 10 according to some embodiments. The operator secures the wound dressings 34, 38, 42 to the wound sites on the patient (process 276). The operator then connects the instillation inlets 98, 98′, 98″ of the wound dressings 34, 38, 42, respectively, to the instillation conduits 110, 110′, 110″, respectively (process 280). The operator then connects the NPWT outlets 102, 102′, 102″ of the wound dressings 34, 38, 42, respectively, to the negative pressure conduits 122, 122′, 122″, respectively (process 284). The operator then connects the wound dressings 34, 38, 42 electronically and pneumatically to the NPWT system 14 and the instillation system 18 (process 288). The operator then enters the treatment parameters for each of the wound sites using the user interface 240 (process 292). The control system 46 then initiates the first instillation therapy treatment and the instillation system controller 256 and the instillation valve controller 264 determine the volume of each of the wound dressings 34, 38, 42 instructed to administer instillation therapy (process 296). The control system 46 then carries out the treatment process based on the treatment parameters input by the user (Process 300). In some embodiments, at least one of the wound dressings 34, 38, 42 can be structured to administer instillation therapy and at least one of the wound dressings 34, 38, 42 can be structured to administer NPWT. Accordingly, the instillation valve controller 264 and the negative pressure valve controller 268 can position the instillation valves 180, 184, 188 and the negative pressure valves 220, 224, 228, respectively, to produce desired instillation and/or NPWT routine specified by the user to each of the wound dressings 34, 38, 42. As the instillation valves 180, 184, 188 are opened to allow instillation fluid to flow into the wound dressings 34, 38, 42, respectively, and/or negative pressure valves 220, 224, 228 are opened to administer negative pressure to the wound dressings 34, 38, 42, respectively, the status indication systems 54, 54′, 54″ indicate the type of wound treatment therapy administered by the wound dressings 34, 38, 42, respectively (Process 304).

### Configuration of Exemplary Embodiments

The construction and arrangement of the systems and methods as shown in the various exemplary embodiments are illustrative only. Although only a few embodiments have been described in detail in this disclosure, many modifications are possible (e.g., variations in sizes, dimensions, structures, shapes and proportions of the various elements, values of parameters, mounting arrangements, use of materials, colors, orientations, etc.). For example, the position of elements can be reversed or otherwise varied and the nature or number of discrete elements or positions can be altered or varied. Accordingly, all such modifications are intended to be included within the scope of the present disclosure. The order or sequence of any process or method steps can be varied or re-sequenced according to alternative embodiments. Other substitutions, modifications, changes, and omissions can be made in the design, operating conditions and arrangement of the exemplary embodiments without departing from the scope of the present disclosure.

The present disclosure contemplates methods, systems and program products on any machine-readable media for accomplishing various operations. The embodiments of the present disclosure can be implemented using existing computer processors, or by a special purpose computer processor for an appropriate system, incorporated for this or another purpose, or by a hardwired system. Embodiments within the scope of the present disclosure include program products comprising machine-readable media for carrying or having machine-executable instructions or data structures stored thereon. Such machine-readable media can be any available media that can be accessed by a general purpose or special purpose computer or other machine with a processor. By way of example, such machine-readable media can comprise RAM, ROM, EPROM, EEPROM, CD-ROM or other optical disk storage, magnetic disk storage or other magnetic storage devices, or any other medium which can be used to carry or store desired program code in the form of machine-executable instructions or data structures and which can be accessed by a general-purpose or special purpose computer or other machine with a processor. Combinations of the above are also included within the scope of machine-readable media. Machine-executable instructions include, for example, instructions and data which cause a general purpose computer, special purpose computer, or special purpose processing machines to perform a certain function or group of functions.

Although the figures show a specific order of method steps, the order of the steps may differ from what is depicted. Also two or more steps can be performed concurrently or with partial concurrence. Such variation will depend on the software and hardware systems chosen and on designer choice. All such variations are within the scope of the disclosure. Likewise, software implementations could be accomplished with standard programming techniques with rule-based logic and other logic to accomplish the various connection steps, processing steps, comparison steps and decision steps.

1. A valve arrangement for administering an instillation and/or negative pressure therapy through at least a first wound dressing and a second wound dressing, the valve arrangement comprising:
an instillation source port structured to engage a instillation fluid source;
a negative pressure source port structured to engage a negative pressure source;
a first instillation flow path including a first instillation valve, the first instillation flow path extending between the instillation source port and a first instillation wound dressing port structured for fluid communication with the first wound dressing;
a second instillation flow path including a second instillation valve, the second instillation flow path extending between the instillation source port and a second instillation wound dressing port structured for fluid communication with the second wound dressing;
a first negative pressure flow path including a first negative pressure valve, the first negative pressure flow path extending between the negative pressure source port and a first negative pressure wound dressing port structured for fluid communication with the first wound dressing; and
a second negative pressure flow path including a second negative pressure valve, the second negative pressure flow path extending between the negative pressure source port and a second negative pressure wound dressing port structured for fluid communication with the second wound dressing.

2. The valve arrangement of claim 1, wherein the first instillation valve and the first negative pressure valve are positionable to allow fluid flow through the first instillation flow path, to allow fluid flow through the first negative pressure flow path, or to allow fluid flow through both the first instillation flow path and the first negative pressure flow path.

3. The valve arrangement of claim 2, further comprised a sensor engaged with the first wound dressing for sensing a condition of the first wound dressing, and wherein a position of at least one of the first instillation valve and the first negative pressure valve is determined based on the condition of the first wound dressing.

4. The valve arrangement of claim 1, wherein the first instillation valve is positionable to administer fluid flow through the first instillation flow path at a first flow rate and the second instillation valve is positionable to administer fluid flow through the second instillation flow path at a second flow rate different than the first flow rate.

5. (canceled)

6. The valve arrangement of claim 4, wherein the first flow rate is based on a volume defined by the first wound dressing and the second flow rate is based on a volume defined by the second wound dressing.

7. The valve arrangement of claim 1, wherein the first negative pressure valve is positionable so that the first wound dressing is placed at a first negative pressure by the negative pressure source and the second negative pressure valve is positionable so that the second wound dressing is placed at a second negative pressure by the negative pressure source, wherein the second negative pressure is different than the first negative pressure.

8. (canceled)

9. The valve arrangement of claim 1, wherein at least one of the first instillation valve, the second instillation valve, the first negative pressure valve, and the second negative pressure valve is continuously positionable between a generally open position and a generally closed position.

10. A valve arrangement for administering an instillation and/or negative pressure therapy through at least a first wound dressing and a second wound dressing, the valve arrangement comprising:
a first flow path extending between a source port and a first wound dressing port structured for fluid communication with the first wound dressing;
a first valve positioned in the first flow path;
a second flow path extending between the source port and a second wound dressing port structured for fluid communication with the second wound dressing; and
a second valve positioned in the second flow path.

11. The valve arrangement of claim 10, wherein the first valve is positionable to allow fluid to flow through the first flow path at a first flow rate and the second valve is positionable to allow the fluid to flow through the second flow path at a second flow rate different than the first flow rate.

12. The valve arrangement of claim 11, wherein at least one of the first flow rate is based on a volume defined by the first wound dressing and the second flow rate is based on a volume defined by the second wound dressing.

13. The system of claim 11, further comprising a sensor engaged with one of the first wound dressing and the second wound dressing, and wherein at least one of the first flow rate and the second flow rate is based on a condition sensed by the sensor.

14. The system of claim 10, wherein at least one of the first valve and the second valve is continuously repositionable between a generally open position and a generally closed position.

15. The system of claim 10, wherein the source port is structured to engage a fluid source and the first valve and the second valve are repositionable to modulate a flow of an instillation source to the first wound dressing and the second wound dressing, respectively.

16. The system of claim 10, wherein the source port is structured to engage a negative pressure source and the first valve and the second valve are repositionable to modulate negative pressure conditions in the first wound dressing and the second wound dressing, respectively.

17. A system, comprising:
a fluid source structured to dispense an instillation fluid;
a negative pressure source structured to generate a negative pressure;
an instillation valve arrangement comprising:
a first instillation flow path extending between an instillation source port structured to engage the fluid source and a first wound dressing port structured to engage a first wound dressing;
a first instillation valve positioned in the first instillation flow path;
a second instillation flow path extending between the instillation source port and a second wound dressing port structured to engage a second wound dressing; and
a second instillation valve positioned in the second instillation flow path;
a negative pressure valve arrangement comprising:
a first negative pressure flow path extending between a negative pressure source port structured to engage the negative pressure source and a first wound dressing port structured to engage the first wound dressing;
a first negative pressure valve positioned in the first negative pressure flow path;
a second negative pressure flow path extending between the negative pressure source port and a second wound dressing port structured to engage the second wound dressing; and
a second negative pressure valve positioned in the second negative pressure flow path.

18. The system of claim 17, wherein the first instillation valve and the first negative pressure valve are repositionable to place the first wound dressing in fluid communication with the fluid source, in fluid communication with the negative pressure source, or in fluid communication with both the fluid source and the negative pressure source.

19. The system of claim 18, wherein the first instillation valve and the first negative pressure valve are positionable to achieve a flow rate through the first wound dressing that is based on a volume defined by the first wound dressing and/or a condition sensed by a sensor positioned within the first wound dressing.

20. The system of claim 18, wherein the second instillation valve and the second negative pressure valve are repositionable to place the second wound dressing in fluid communication with the fluid source, in fluid communication with the negative pressure source, or in fluid communication with both the fluid source and the negative pressure source.

21. The system of claim 20, wherein the first instillation valve and the first negative pressure valve are controlled independently of the second instillation valve and the second negative pressure valve.

22. The system of claim 17 further comprising a controller in electrical communication with the first instillation valve and the second instillation valve and a sensor engaged with at least one of the first wound dressing and the second wound dressing, and wherein the controller is structured to close the first instillation valve in response to determining that the first wound dressing is full and structured to close the second instillation valve in response to determining that the second wound dressing is full.

23. The system of claim 17, wherein at least one of the first instillation valve, the second instillation valve, the first negative pressure valve, and the second negative pressure valve is continuously positionable between a generally open position and a generally closed position.

24. (canceled)

25. (canceled)

26. (canceled)

27. (canceled)

28. (canceled)

29. (canceled)
